(19) United States
(12) Patent Application Publication
(10) Pub. No.: US 2008/0065001 A1
(43) Pub. Date: Mar. 13, 2008

(54) Portable debridement and irrigation device

(76) Inventors: Kent DiNucci, Omaha, NE (US); Dennis Gusman, Enumclaw, WA (US)

Correspondence Address: LAW OFFICES OF ADAM H. JACOBS, Suite 726, 1904 Farnam Street, Omaha, NE, 68102, US

(21) Appl. No.: 11/508,796

(22) Filed: Aug. 23, 2006

(51) Int. Cl. A61N 1/30

(52) U.S. Cl. 604/19; 239/373; 239/288; 604/140; 604/73

(57) ABSTRACT

A portable debridement and irrigation device includes a body structure and a disposable spray nozzle structure and spray reduction cup removably mounted thereon. A battery-powered electric pump device is in liquid transmission connection with the disposable spray nozzle device. A removable and replaceable liquid storage container which holds a sterile liquid is held within a storage container receiving chamber formed within the body structure in liquid transmission connection with the electric pump, and a pump engagement switch is operatively interposed between the battery and the electric pump for engaging and disengaging the electric pump. Finally, a pump speed control device with a limiting device for preventing it from proceeding beyond a predetermined operating setting unless the operator intentionally disengages the limiting device is operatively connected to the electric pump for adjusting the operating speed of the pump and thus the outflow speed of liquid from the spray nozzle.

### BACKGROUND OF THE INVENTION

1. Technical Field

The present invention is directed to debridement devices for cleaning and irrigating flesh wounds and, more particularly, to a portable debridement and irrigation device which includes a body structure, a disposable spray nozzle removably mounted on the body structure, a pump mounted within the body structure in liquid transmission connection with the disposable spray nozzle, a removable and replaceable liquid storage container which fits within a storage container receiving chamber within the body structure such that liquid held within the liquid storage container may be removed by operation of the pump and dispersed through the spray nozzle, and a pump speed control device operatively connected to the pump for adjustably controlling the operating speed of the pump and hence the outflow speed of liquid from the disposable spray nozzle, the pump speed control device also including a limiting device operative to prevent the pump speed control from proceeding beyond a predetermined operating setting unless the operator intentionally disengages the limiting device to permit the pump speed control device to proceed beyond the predetermined operating setting to increase the operating speed of the pump.

2. Description of the Prior Art

Prompt and efficient care of flesh wounds is vital to prevent additional tissue damage and even more importantly to prevent secondary infection which will aggravate the wound and significantly increase the recovery time needed to recover from the injury. One of the most important elements of care is to properly clean and treat the wound prior to dressing it to begin the healing process. In general, the cleaning process involves flushing or irrigating the wound with sterile water or saline solution to both remove any debris from the wound and also remove any harmful bacteria from the wound. For example, many victims of vehicular accidents have wounds which have gravel, sand and other contaminants in them from contact with the road, and it is a necessary step in the healing process to thoroughly clean the wound of these materials prior to dressing the wound. This process is ordinarily performed with manual squeeze bottles or the like which are used by the nurses and doctors to spray out the wound and remove as much debris as possible from the wound area. However, there is a significant problem encountered in using such manual squeeze bottles, and this is that the strength of the liquid flow is dependent on the strength of the individual using the squeeze bottle, and it is entirely possible that an insufficient amount of spraying force will be applied to the wound, thus improperly cleaning the wound area.

Several devices have been proposed in the prior art which attempt to address and solve this problem. These include such devices as Arnett et al., U.S. Pat. No. 6,022,329, and a currently pending PCT application, specifically Bidoia, PCT Serial No. WO2006040273, which each disclose surgical irrigation devices which may be used to irrigate a wound to cleanse and debride the wound. However, both of these devices have inherent deficiencies, specifically that they are not portable and not easily manipulated to clean the wound, and furthermore each require separate associable reservoirs for cleaning liquid which render both of these devices non-portable. There is therefore a need for a portable debridement and irrigation device which may be easily used by nurses, doctors, and paramedics for quickly and efficiently debriding and irrigating a wound and which does not rely on external liquid reservoirs or power supplies for operation thereof.

Therefore, an object of the present invention is to provide an improved portable debridement and irrigation device.

Another object of the present invention is to provide a portable debridement and irrigation device which includes easily removable and replaceable liquid storage containers which help to ensure a sterile and renewable liquid supply for the device.

Another object of the present invention is to provide a portable debridement and irrigation device which includes a portable power source such as batteries which are mounted within the body structure of the device to enhance the portability of the device.

Another object of the present invention is to provide a portable debridement and irrigation device which includes a disposable spray nozzle section having a spray reduction cup mounted thereon, the disposable spray nozzle section capable of being quickly and easily separated from the body structure of the device and disposed of after use.

Another object of the present invention is to provide a portable debridement and irrigation device which includes a pump speed control device operative to enable adjustment of the speed of the pump to increase or decrease the force with which water or another such liquid is propelled from the spray nozzle to provide a controllable and efficient device for debriding and irrigating wounds.

Finally, an object of the present invention is to provide a portable debridement and irrigation device which is relatively simple in design and construction and is safe, effective, and efficient in use.

### SUMMARY OF THE INVENTION

The present invention provides a portable debridement and irrigation device including a body structure and a disposable spray nozzle structure removably mounted on the body structure. A spray reduction cup is mounted on and at least partially surrounds the disposable spray nozzle device for generally confining spray from the disposable spray nozzle device and an electric pump device is mounted within the body structure in liquid transmission connection with the disposable spray nozzle device. A power device such as a battery is mounted within the body structure in power transmission connection with the electric pump, and a removable and replaceable liquid storage container which holds a sterile liquid such as saline solution is held within a storage container receiving chamber formed within the body structure. A liquid storage container engagement is mounted within the storage container receiving chamber for engaging the liquid storage container upon the liquid storage container being inserted into the storage container receiving chamber for removal of liquid stored therein, the liquid storage container engagement means in liquid transmission connection with the electric pump, and a pump engagement switch is operatively interposed between the power means and the electric pump for engaging and disengaging the electric pump. Finally, a pump speed control device is operatively connected to the electric pump for adjusting the operating speed of the pump and thus the outflow speed of liquid from the disposable spray nozzle with a limiting device being operatively connected to the pump speed control device for preventing the pump speed control device from proceeding beyond a predetermined operating setting unless the operator intentionally disengages the limiting device whereby the pump speed control device is permitted to proceed beyond the predetermined operating setting thereby increasing the operating speed of the pump and hence the outflow speed of liquid from the spray nozzle.

It is therefore abundantly clear that the portable debridement and irrigation device of the present invention includes many valuable features not found in the prior art. For example, because the liquid storage containers are removable and replaceable within the device, the sterility of the liquid being used to debride and irrigate the wound is virtually guaranteed. Furthermore, because the present invention includes a pump speed control device which permits quick and simple adjustment of the strength of the jet of sterile water or other such liquid emanating from the spray nozzle, virtually any type of wound may be cleaned by using the present invention, while ensuring a high degree of accuracy and reducing the possibility of creating further damage in the wound. Also, because the present invention is entirely portable, it is ideally suited for use by paramedics and other field workers who need to quickly and efficiently debride and irrigate wounds and even afflicted external organs such as eyes, noses and mouths prior to transporting the injured person to a medical care facility. It is therefore clear that the present invention provides a substantial improvement over those debridement and irrigation devices found in the prior art.

### BRIEF DESCRIPTION OF THE DRAWINGS

FIG. 1 is a perspective view of the portable debridement and irrigation device of the present invention being used to debride and irrigate a flesh wound;

FIG. 2 is a perspective view of the invention;

FIG. 3 is a detailed perspective view showing the disconnecting of the spray nozzle from the body of the device;

FIG. 4 is a perspective view of the present invention showing the position of the removable and replaceable liquid storage container within the device;

FIG. 5 is a side elevational view showing the insertion of the liquid storage container into the device; and

FIG. 6 is a detailed perspective view showing the internal working elements of the invention.

### DESCRIPTION OF THE PREFERRED EMBODIMENT

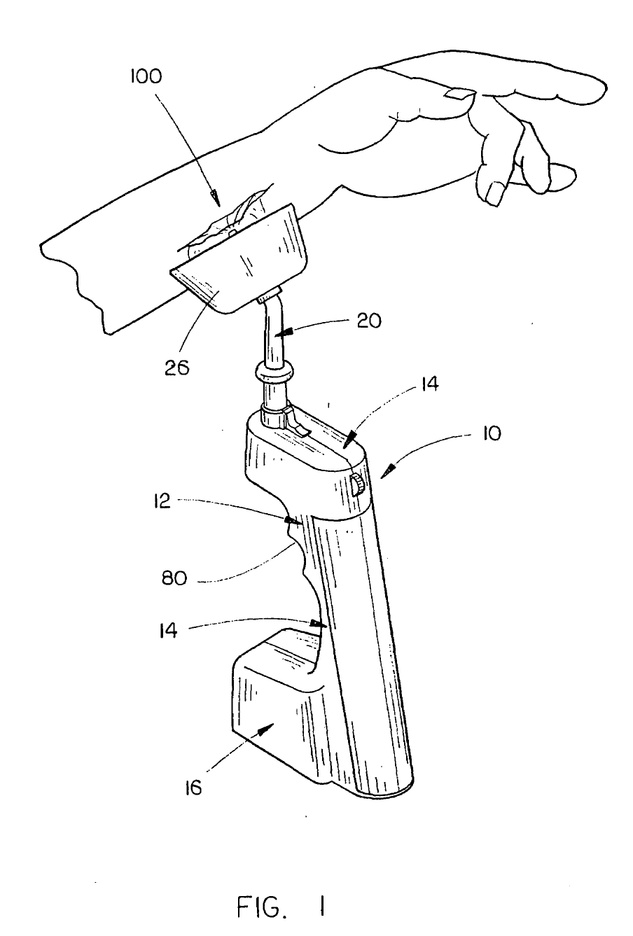

The portable debridement and irrigation device 10 of the present invention is shown best in FIGS. 1-6 as including a generally hollow body structure 12 which is generally C-shaped when viewed from the side and which includes an upper section 14, handle section 16, and battery enclosure section 18. In the preferred embodiment, the body structure 12 would preferably be constructed of a molded plastic material which is relatively light in weight yet is wear and damage resistant, and the body structure 12 may be generally of any appropriate color, size or shape, although the shape as shown in the accompanying figures is generally preferred.

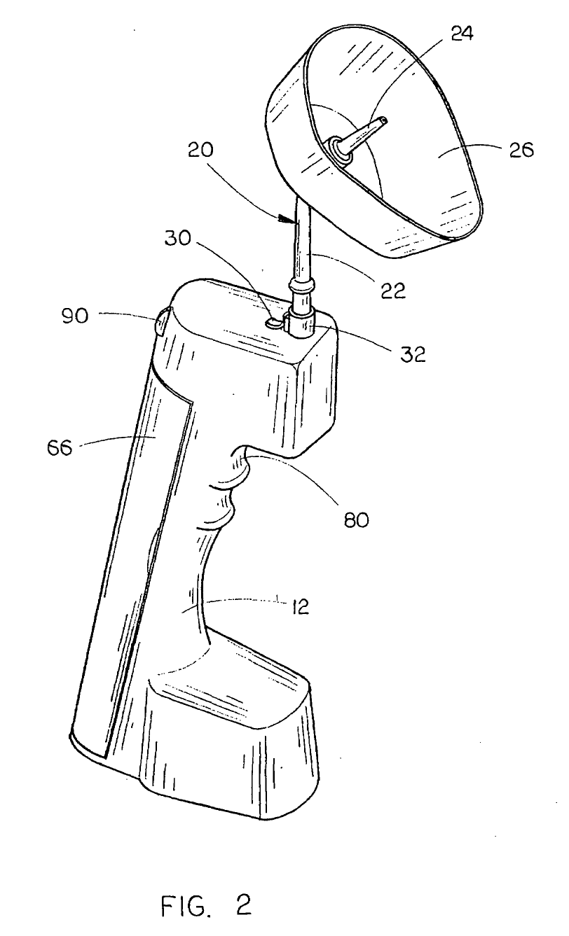

Removably mounted atop upper section 14 of body structure 12 is the spray nozzle assembly 20 which includes a generally upright hollow shaft 22 on top of which is mounted spray nozzle 24, as shown best in FIG. 2. To facilitate use of the present invention, it is generally preferred that spray nozzle 24 be angled approximately thirty to sixty degrees from vertical alignment with the upright hollow shaft 22, although the specific angle at which spray nozzle 24 extends from upright hollow shaft 22 is not particularly critical to the present invention.

Mounted on and generally surrounding spray nozzle 24 of spray nozzle assembly 20 is a spray reduction cup 26 which, in the preferred embodiment, would be a hollow plastic cup mounted on the top of upright hollow shaft 22 or the base of spray nozzle 24 which generally surrounds and encloses the spray nozzle 24, as shown best in FIGS. 1 and 2. The spray reduction cup 26 is useful in that it acts to generally prevent back spray from the impact of the water jet with the wound area being cleansed, as the back spray is caught by the spray reduction cup 26 and redirected downwards within the spray reduction cup 26. This is of particular importance when the wound being cleaned is rife with pathogens which may be communicable, and the spray resulting from impact of the water jet with the wound area may cause communication of the pathogens to health care workers in the immediate vicinity. It has been found that the spray reduction cup 26 substantially reduces the likelihood of airborne pathogens caused by water impact with the wound area, and therefore its inclusion is considered important to the present invention. However, the exact size and shape of the spray reduction cup 26 is not critical to the present invention so long as the spray reduction cup 26 performs its intended function of generally preventing back spray and scatter spray from the impact of the water jet with the wound area.

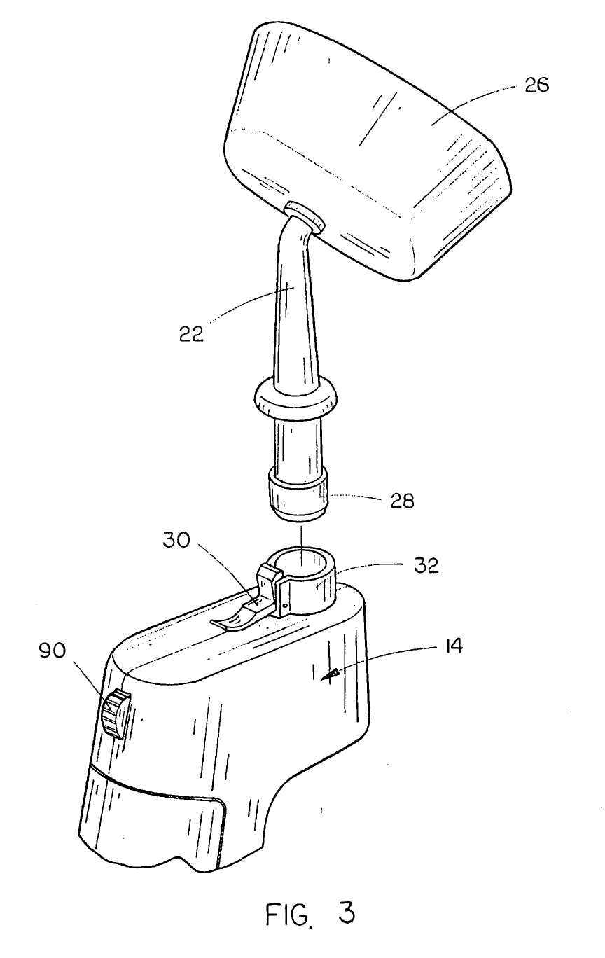

An important feature of the present invention is that the spray nozzle assembly 20 is removably and releasably mounted on the upper section 14 of body structure 12 by a latch 30, such as that shown in FIGS. 2 and 3. In the preferred embodiment, an annular flange 28 formed surrounding the base of the upright hollow shaft 22 would be slid into a generally cylindrical mounting ring 32 which is mounted on top of upper section 14 of body structure 12. The fit between the annular flange 28 and mounting ring 32 is generally liquid-tight once the hollow shaft 22 is seated within the mounting ring 32. Once the annular flange 28 is seated fully within the mounting ring 32, latch 30 engages the top of annular flange 28 to releasably secure the spray nozzle assembly 20 within the mounting ring 32 and hence on upper section 14 of body structure 12. Of course, many other types of connections and latch assemblies may be used in connection with the present invention to secure the spray nozzle assembly 20 on the body structure 12, but it has been found that the assembly as thus described provides a simple and efficient mechanical connection between the spray nozzle assembly 20 and upper section 14 of body structure 12.

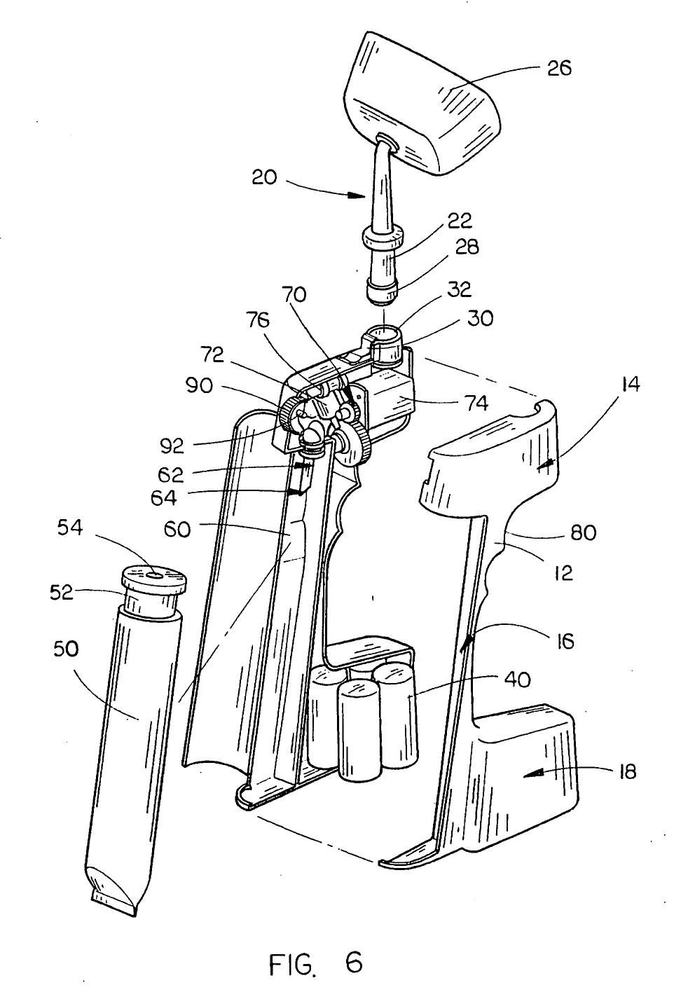

FIG. 6 illustrates the internal workings of the portable debridement and irrigation device 10 of the present invention, including the positioning of the batteries 40 within the battery enclosure section 18 of body structure 12. Of course, the actual size, shape, voltage and type of batteries 40 used in connection with the present invention may be modified or changed, but it is preferred that a nickel cadmium or lithium ion rechargeable battery is best suited for use with the present invention to provide power to the unit. Accordingly, the size and shape of the battery enclosure section 18 may be modified and changed depending on the shape of the batteries 40 used in connection with the present invention.

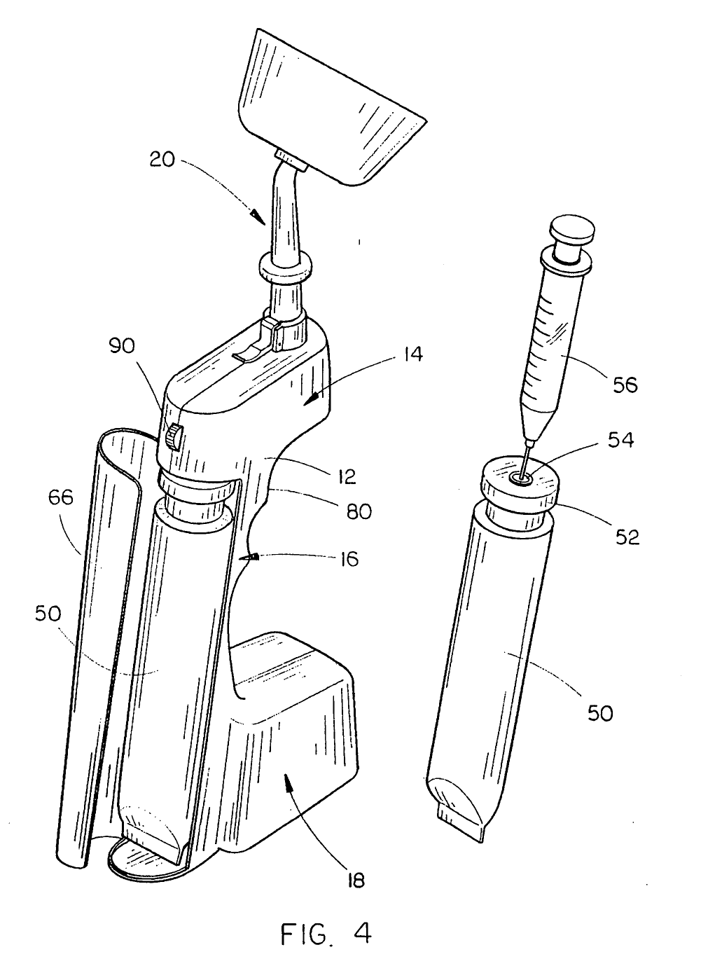

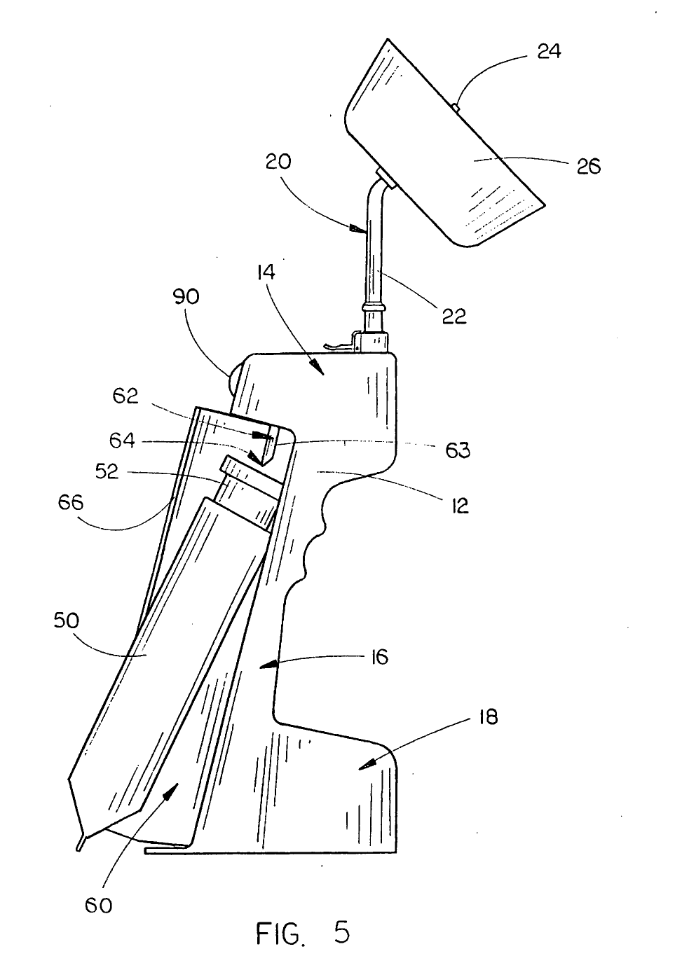

FIGS. 4 and 5 illustrate an important element of the present invention, specifically the removable and replaceable liquid storage container 50 which fits within the liquid storage container receiving chamber 52 formed within the body structure 12 of portable debridement and irrigation device 10, specifically within handle section 16 thereof. In the preferred embodiment, the liquid storage container 50 would preferably be constructed of a flexible plastic material having a generally cylindrical shape which includes an upper container stopper 52 having a central penetrable rubber plug 54 which, when penetrated, permits the liquid stored within the liquid storage container 50 to be released therethrough. In the preferred embodiment, the liquid storage container 50 would be approximately eight to twelve inches (8″ to 12″) in length and one to two inches (1″ to 2″) in diameter, and the liquid stored therein would be a sterile saline or water solution, depending on the irrigation needs of the user of the present invention. Also, the liquid storage container 50 can have various medicines or antibiotics added to the liquid stored therein, as shown in FIG. 5, by merely injecting the additive into the liquid storage container 50 via a syringe 54 or the like without damaging the sterile nature of the liquid held therewithin, an improvement which is not demonstrated in the prior art.

Mounted within and extending downwards from the top of the storage container receiving chamber 60 is a liquid storage container engagement device 62 which, in the preferred embodiment, would be a hollow tube 63 having a sharpened penetrating end 64 which is designed to engage the plug 54 on liquid storage container 50 and penetrate the plug 54 such that the hollow tube 63 extends into the liquid storage container 50. Because the seal between the penetrating end 64 of hollow tube 63 and plug 54 of liquid storage container 50 is substantially fluid-tight, all of the liquid housed within the liquid storage container 50 is removable through the hollow tube 63 for use by the portable debridement and irrigation device 10 of the present invention. Once the liquid storage container 50 is emptied, it then is then a simple matter to reverse the insertion procedure by sliding the liquid storage container 50 out of the storage container receiving chamber 60 and replacing the liquid storage container 50 with a new liquid storage container 50 which is full of sterile liquid. Finally, a storage container receiving chamber door 66 is provided to secure the liquid storage container 50 within the storage container receiving chamber 60, as shown best in FIGS. 1 and 2.

The portable debridement and irrigation device 10 of the present invention further includes an electric pump 70 which is mounted within the upper section 14 of body structure 12, as shown best in FIG. 6. The electric pump 70 may be of many different sizes, shapes and capacities, although it is preferred that the electric pump 70 be able to provide a generally continuous stream of liquid to the spray nozzle assembly 20 for distribution through the spray nozzle 24 towards the wound area 100 in a steady, focused stream. The electric pump 70 may consist of a pumping device 72 and an electric motor 74, wherein the pumping device 72 is in liquid transmission connection with the container engagement device 62 of storage container receiving chamber 60 and the upright hollow shaft 22 of spray nozzle assembly 20. Specifically, the pumping device 72 functions in the following manner: Once the liquid storage container 50 is penetrated by the penetrating end 64 of container engagement device 62, liquid from the liquid storage container 50 flows through the hollow tube of container engagement device 62 towards the pumping device 72. When the pumping device is activated, this liquid is drawn out of the liquid storage container 50, accelerated, and driven into liquid tube 76 which connects with the upright hollow shaft 22 of spray nozzle assembly 20 when the spray nozzle assembly 20 is mounted within the mounting ring 32 on upper section 14 of body structure 12. The liquid is thus forced out of the spray nozzle 24 towards the would area 100 in a focused stream or spray, depending on the type and shape of the nozzle, thus performing the intended debridement and irrigation of the wound area 100.

Power for the electric motor 74 is, of course, provided by the batteries 40 which are connected to the electric motor 74 by a power transmission cable (not shown), and a trigger switch 80 or the like is provided to engage and disengage the electric motor 74 of electric pump 70 thereby driving the pumping device 72. In the preferred embodiment, the trigger switch 80 would be mounted on the forward portion of the handle section 16 for easy engagement by the index or middle finger of the individual holding the portable debridement and irrigation device 10 of the present invention, although the precise location of the trigger switch 80 on the body structure 12 is not critical to the present invention so long as it is convenient to operate.

One of the additional important features of the present invention is that the operating speed of the electric pump 70 is adjustable via the pump speed control device 90 which is mounted on the rear surface of the upper section 14 of the body structure 12, as shown best in FIGS. 1 and 6. In the preferred embodiment, the pump speed control device 90 would be a rotatable wheel operatively connected to an adjustable rheostat or resistor which adjusts the operational speed of the electric pump 70 by controlling either the rotational speed of the shaft of the electric motor 74 by limiting the current being passed to the electric motor 74 or by physically retarding the rotation of the shaft of the electric motor 74. Alternatively, however, the pump speed control device 90 could be any other type of adjustment device, such as a lever, knob, sliding button or other such device, so long as the pump speed control device 90 is operative to adjust the operational speed of the electric pump 70 or at least the flow speed of the water jet emanating from the spray nozzle 24.

An important feature of the pump speed control device 90, however, is that it in actuality includes two separate speed regimes, the first being the slow or low speed regime which is designed to irrigate and partially debride the wound area 100 and the high speed regime which is designed to fully debride the wound area 100 but which may also cause damage to the surrounding tissues of the wound area 100. The pump speed control device 90 therefore includes a limiting device 92 which prevents the pump speed control device 90 from entering the high speed regime unless the operator of the device intentionally disengages the limiting device 92. In practice, this will likely be done by providing a mechanical limiting device which permits the rotation of the pump speed control device 90 to and through a selected set of operating settings with a maximum setting being preset by the operator or by the manufacturer, and once the limiting device 92 is disengaged, the pump speed control device 90 may be rotated through its entire range of operational settings. The purpose for this limiting device 92 is to prevent the health care specialist from accidentally causing additional damage to the wound area 100 as may be caused by a high-pressure water jet impacting already damaged tissue. It has been found that higher pressure water jets impacting already damaged tissue may cause additional damage beyond that caused by the initial injury, and while in some cases it is absolutely necessary to remove dead or dying tissue from the area of the wound, it will be at the discretion of the health care professional to disengage the limiting device 92 to provide access to the high speed regime of the present invention. In normal circumstances, however, it is important to limit the speed and impact force of the water jet emanating from the spray nozzle 24 to generally prevent additional damage to the wound area 100.

In operation, the portable debridement and irrigation device 10 would be used in the following manner. The user would insert a liquid storage container 50 into the storage container receiving chamber 60 and penetrate the plug 54 such that the hollow tube 63 extends into the liquid storage container 50. The batteries 40 would be at operating capacity, and the spray nozzle 24 would be aimed at the wound area 100, as shown in FIG. 1. The trigger switch 80 would then be engaged to activate the electric pump 70 and sterile liquid would be drawn from the liquid storage container 50 and sent to the spray nozzle 24 to be directed onto the wound area 100 for cleaning thereof. The pump speed control device 90 would be adjusted, and a decision on disengaging the limiting device 92 would also be made by the operator of the invention. Finally, once the wound area 100 has been cleaned, the trigger switch 80 would be disengaged and the water jet emanating from the spray nozzle 24 would be stopped. The portable debridement and irrigation device 10 of the present invention is then ready for further use thereof.

A further important use of the present invention would involve small modifications to the spray nozzle 24 and the spray reduction cup 26, namely that instead of a directed jet of liquid coming from the spray nozzle 24 during operation of the device, the spray nozzle 24 would instead send a diffused spray out therefrom, and the spray reduction cup 26 would be modified in shape such that the spray reduction cup 26 fits snugly over the eye socket of a user of the present invention. This allows the present invention to be used as the first truly portable eye wash device, and provides a significant improvement over all other prior art eye wash devices. Because the present invention is completely portable, it now becomes unnecessary for a person who has injured their eye to go to the eye wash station, as the eye wash station can effectively come to the person in the form of the modified present invention. One would need merely place the modified spray reduction cup 26 over the afflicted eye and engage the trigger switch 80 to start the flow of sterile liquid out of the spray nozzle 24. As the spray nozzle 24 has been modified to output a diffuse spray, the eye is washed out with the sterile liquid and therefore immediate care is provided to the afflicted eye. It is not necessary to move the individual being treated from his or her particular location in order to treat their eye injury, and this will facilitate treatment of the injury. It is therefore seen that the present invention provides a substantial improvement over not only other portable debridement devices but also other eye wash devices found in the prior art.

It is to be understood that numerous additions, modifications and substitutions may be made to the portable debridement and irrigation device 10 of the present invention which fall within the intended broad scope of the appended claims. For example, the size, shape and construction materials used in connection with the device may be modified or changed depending on the needs and desires of the users of the invention, and such modifications should be understood to be a part of this disclosure so long as they do not significantly adversely impact the functional characteristics of the present invention. Furthermore, the precise size, shape and design of the liquid storage containers 50 is not critical to the present invention so long as they are capable of carrying sterile liquids and liquid solutions and may be quickly and easily removed from and replaced within the storage container receiving chamber 60 of the present invention. Finally, although the pump speed control device 90 and limiting device 92 have been described herein with some particularity, many different types of speed control devices and limiting devices may be used with the present invention so long as the intended functional features of those elements are generally preserved.

There has therefore been shown and described a portable debridement and irrigation device 10 which accomplishes at least all of its intended objectives.

I claim:

1. A portable debridement and irrigation device comprising:
a body structure;
disposable spray nozzle means removably mounted on said body structure;
spray reduction cup means mounted on and at least partially surrounding said disposable spray nozzle means for generally confining spray from said disposable spray nozzle means; pump means mounted within said body structure in liquid transmission connection with said disposable spray nozzle means;
power means mounted within said body structure in power transmission connection with said pump means;
a removable and replaceable liquid storage container;
a storage container receiving chamber within said body structure for receiving and releasably retaining said liquid storage container therein;
liquid storage container engagement means mounted within said storage container receiving chamber for engaging said liquid storage container upon said liquid storage container being inserted into said storage container receiving chamber for removal of liquid stored therein, said liquid storage container engagement means in liquid transmission connection with said pump means;
pump engagement means operatively interposed between said power means and said pump means for engaging and disengaging said pump means;
pump speed control means operatively connected to said pump means for adjusting the operating speed of said pump means and thus the outflow speed of liquid from said disposable spray nozzle means; and
limiting means operatively connected to said pump speed control means for preventing said pump speed control means from proceeding beyond a predetermined operating setting unless the operator intentionally disengages said limiting means whereby said pump speed control means is permitted to proceed beyond the predetermined operating setting thereby increasing the operating speed of said pump means.

2. The portable debridement and irrigation device of claim 1 wherein said disposable spray nozzle means is mounted on said body structure by a latch means operative to releasably secure said disposable spray nozzle means on said body structure.

3. The portable debridement and irrigation device of claim 1 wherein said spray reduction cup means extends around and substantially surrounds said disposable spray nozzle means.

4. The portable debridement and irrigation device of claim 1 wherein said pump means comprises an electric pump.

5. The portable debridement and irrigation device of claim 1 wherein said liquid storage container comprises a generally cylindrical container constructed of a flexible plastic material, said liquid storage container further including an upper container stopper having a central penetrable rubber plug which, when penetrated, permits the liquid stored within said liquid storage container to be released therethrough.

6. The portable debridement and irrigation device of claim 5 wherein said liquid storage container engagement means comprises a hollow tube extending into said storage container receiving chamber, said hollow tube having a sharpened penetrating end adapted to engage said central penetrable rubber plug on said liquid storage container and penetrate said central penetrable rubber plug such that said hollow tube extends into said liquid storage container whereby all of the liquid housed within said liquid storage container is removable through said hollow tube.

7. The portable debridement and irrigation device of claim 1 wherein said pump speed control means comprises one of an adjustable rheostat and resistor operative to control the operational speed of said pump means by controlling the operational speed of said pump means by one of limiting the amount of electric current being transmitted to said pump means and by physically retarding operation of said pump means.

8. The portable debridement and irrigation device of claim 1 wherein said limiting means comprises a mechanical limiting device operative to permit operation of said pump speed control means to and through a selected set of operating settings with a lower maximum setting being preset, said limiting means operative to permit said pump speed control means to be operated through its entire range of operational settings upon said limiting device being disengaged.

9. The portable debridement and irrigation device of claim 1 wherein said disposable spray nozzle means and said spray reduction cup means are modified such that said disposable spray nozzle means outputs a diffused spray and said spray reduction cup means is adapted to fit snugly over the eye socket of a user of the present invention whereby said portable debridement and irrigation device is usable as a portable eye wash device.

10. A portable debridement and irrigation device comprising:
a body structure;
disposable spray nozzle means removably mounted on said body structure;
spray reduction cup means mounted on and at least partially surrounding said disposable spray nozzle means for generally confining spray from said disposable spray nozzle means; an electric pump mounted within said body structure in liquid transmission connection with said disposable spray nozzle means;
power means mounted within said body structure in power transmission connection with said pump means;
a removable and replaceable liquid storage container containing a quantity of sterile liquid;
a storage container receiving chamber within said body structure for receiving and releasably retaining said liquid storage container therein;
liquid storage container engagement means mounted within said storage container receiving chamber for engaging said liquid storage container upon said liquid storage container being inserted into said storage container receiving chamber for removal of said sterile liquid stored therein, said liquid storage container engagement means in liquid transmission connection with said electric pump;
pump engagement means operatively interposed between said power means and said pump means for engaging and disengaging said pump means;
pump speed control means operatively connected to said pump means for adjusting the operating speed of said pump means and thus the outflow speed of liquid from said disposable spray nozzle means; and
limiting means operatively connected to said pump speed control means for preventing said pump speed control means from proceeding beyond a predetermined operating setting such that said electric pump operates in a lower operating speed regime, said limiting means further operative such that upon said limiting means being disengaged, said pump speed control means is permitted to proceed beyond the predetermined operating setting thereby permitting said electric pump to operate in both said lower operating speed regime and in an upper operating speed regime.

11. The portable debridement and irrigation device of claim 10 wherein said disposable spray nozzle means is mounted on said body structure by a latch means operative to releasably secure said disposable spray nozzle means on said body structure.

12. The portable debridement and irrigation device of claim 10 wherein said spray reduction cup means extends around and substantially surrounds said disposable spray nozzle means.

13. The portable debridement and irrigation device of claim 10 wherein said liquid storage container comprises a generally cylindrical container constructed of a flexible plastic material, said liquid storage container further including an upper container stopper having a central penetrable rubber plug which, when penetrated, permits the liquid stored within said liquid storage container to be released therethrough.

14. The portable debridement and irrigation device of claim 10 wherein said liquid storage container engagement means comprises a hollow tube extending into said storage container receiving chamber, said hollow tube having a sharpened penetrating end adapted to engage said central penetrable rubber plug on said liquid storage container and penetrate said central penetrable rubber plug such that said hollow tube extends into said liquid storage container whereby all of the liquid housed within said liquid storage container is removable through said hollow tube.

15. The portable debridement and irrigation device of claim 10 wherein said pump speed control means comprises one of an adjustable rheostat and resistor operative to control the operational speed of said pump means by controlling the operational speed of said pump means by one of limiting the amount of electric current being transmitted to said pump means and by physically retarding operation of said pump means.

16. The portable debridement and irrigation device of claim 10 wherein said limiting means comprises a mechanical limiting device operative to permit operation of said pump speed control means to and through a selected set of operating settings with a lower maximum setting being preset, said limiting means operative to permit said pump speed control means to be operated through its entire range of operational settings upon said limiting device being disengaged.

17. The portable debridement and irrigation device of claim 10 wherein said disposable spray nozzle means and said spray reduction cup means are modified such that said disposable spray nozzle means outputs a diffused spray and said spray reduction cup means is adapted to fit snugly over the eye socket of a user of the present invention whereby said portable debridement and irrigation device is usable as a portable eye wash device.
